(19) United States
(12) Patent Application Publication
(10) Pub. No.: US 2009/0300867 A1
(43) Pub. Date: Dec. 10, 2009

(54) CLEAN BUTTON

(76) Inventor: Behrouz Farhoudi, Zaandam (NL)

Correspondence Address: DICKINSON WRIGHT PLLC, 38525 WOODWARD AVENUE, SUITE 2000, BLOOMFIELD HILLS, MI, 48304-2970, US

(21) Appl. No.: 12/307,050

(22) PCT Filed: Jul. 4, 2007
(86) PCT No.: PCT/NL07/50329

§ 371 Date: Jul. 8, 2009

(30) Foreign Application Priority Data
Jul. 4, 2006 (NL) 2000125

(51) Int. Cl. A61F 13/38

(52) U.S. Cl. 15/209.1

(57) ABSTRACT

A cleaning swab comprising a handle which is provided at at least one end thereof with a thickened portion consisting of absorption material, wherein the handle includes a recess near the end provided with the thickened portion, with the absorption material covering the front edge of the recess, leaving the entrance to the recess open, wherein the handle comprises at least two handle segments, and wherein the handle segment that faces towards the thickened portion deforms more easily than the handle segment that faces away from the thickened portion.

The invention relates to a cleaning swab comprising a handle which is provided at at least one end thereof with a thickened portion consisting of absorption material.

Such a cleaning swab provided with a thickened portion is generally known, it is used in particular for cleaning ears, that is, for removing in particular earwax from ears. In practice the known cleaning swab appears to have the disadvantage that the earwax cannot always be easily removed from the ear, which may induce some people to attempt to scrape the earwax from the ear by means of sharp objects, with all the harmful consequences this may have, such as damage to the ear canal.

The object of the invention is to improve the known cleaning swab, in particular in the sense that dirt, moisture and earwax can be easily removed from the ear without any risk of damage to the ear.

In order to accomplish that object, a cleaning swab of the kind referred to in the introduction is characterised in that the handle includes a recess near the end provided with the thickened portion, with the absorption material covering the front edge of the recess, leaving the entrance to the recess open, wherein the handle comprises at least two handle segments, and wherein the handle segment that faces towards the thickened portion deforms more easily than the handle segment that faces away from the thickened portion. Preferably, the absorption material extends over the front edge and into the recess. The invention is in particular based on the perception that earwax can be efficiently transferred to the recess by alternately pushing the present cleaning swab into the ear canal and rotating it, whilst damage to the ear canal is prevented in that the front edge of the recess is covered with the absorption material. The absorption material is in particular capable of absorbing moisture present in the ear canal. The segmented configuration of the handle furthermore helps to have said cleaning take place in a safe manner, without any risk of causing damage, for example to the ear canal.

It should be noted that the phrase “deforms more easily” is in particular understood to mean that if a harmful load is exerted on the cleaning swab, the handle segment that faces towards the thickened portion will deform sooner than the handle segment that faces away from said thickened portion. This may be achieved by making said segment less stiff, but in some cases also by making it less strong but stiffer, so that the handle segment that faces towards the thickened portion will nevertheless deform sooner when a harmful threshold load is exerted thereon, thus preventing the exertion of a high, local load.

The present cleaning swab can be produced at low cost, using simple production means.

It is noted that the invention is not limited to a cleaning swab for cleaning ears, but that it also extends to other applications, such as cleaning other body cavities, for example the oral cavity, cosmetic applications, for example applying makeup, or medical applications, for example cleaning wounds or applying an ointment or the like.

In a preferred embodiment of a cleaning swab according to the invention, the absorption material comprises at least one material selected from the group consisting of cotton, wool, paper and plastic.

In another preferred embodiment of a cleaning swab according to the invention, the handle is provided with an internal channel that forms the recess at the end of the handle provided with the thickened portion. The channel may in principle have any cross-sectional shape, such as a round or angular shape, so that the channel does not necessarily have to be tubular or cylindrical in shape.

In a preferred embodiment of a cleaning swab according to the invention, the handle segments are made of different materials and the material of the handle segment that faces towards the thickened portion deforms more easily than the material of the handle segment that faces away from the thickened portion. The handle segments are preferably glued together. In another preferred variant, said handle segments have been thermally bonded together. The handle segments are preferably tubular segments.

It is noted that the segmentation of the handle can also be realised by locally subjecting the handle to a treatment, for example a chemical or physical treatment, assuming that the handle is made of one starting material. For example, a strip of starting material of paper/cardboard may be provided with weakened portions, such as perforations, cuts and the like near an edge zone, so that handle segments mutually differing in strength are formed after rolling up the strip into a handle. The shape of the perforations, for example, may be used for checking the deformability. In another preferred variant, segmentation of the handle can be realised by providing the handle with cuts near the front edges of the recess (whether or not at a specific angle relative to the longitudinal axis of the handle), so that said end of the handle thus configured as a bundle of coarse fibres will be more flexible.

In another preferred embodiment of a cleaning swab according to the invention, the recess includes a cutting profile. Although the cutting profile may in principle have any cross-sectional shape, it is preferably thread-like, with the threads of the cutting profile being stretched over the opening of the recess at or near the front edges thereof, which threads may be attached to or near said front edges, for example by gluing or by means of locally provided holes and cuts. As a result, especially earwax can be transferred to the recess by carrying out a certain cutting or scraping action, such that this nevertheless takes place in a safe manner.

In a preferred embodiment of a cleaning swab according to the invention, the handle comprises a stop surface that functions to prevent the handle from being inserted to deep into a body cavity. The stop surface is in particular detachably connected to the handle, preferably by means of a snap connection. In this way the stop surface can be easily snapped onto and be removed from the handle.

In another preferred embodiment of a cleaning swab according to the invention, the handle is provided with a recess and with a thickened portion of absorption material near both ends, with the absorption material covering the front edges of the recesses, leaving the entrance to the recesses open. Preferably, the absorption material extends over the front edge and into the recess. In other words, the handle is provided with two depths, which makes it easier to use.

It is noted that German patent DE 196 38 913 discusses a cleaning swab provided with a thickened portion of plastic material having a serrated cross-sectional shape, wherein. earwax is removed from the ear canal by making use of the sharp edges of the serrated shape, with all the consequent risks of damage to the ear.

The invention will not be explained in more detail with reference to figures illustrated in a drawing, in which

FIGS. 1-3 are longitudinal sections of different preferred variants of a cleaning swab according to the invention;

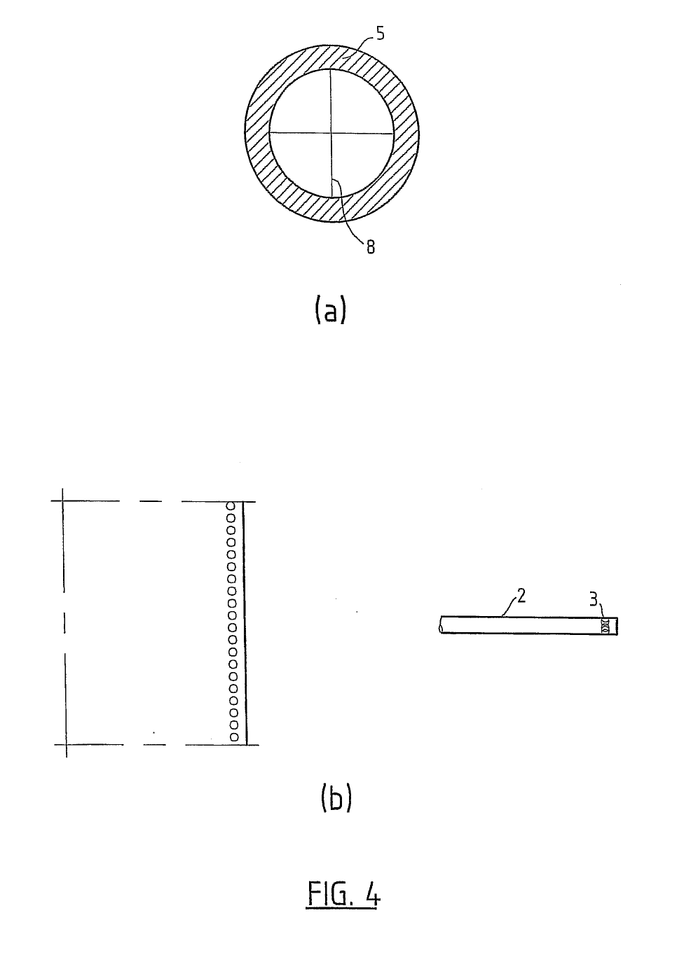

FIG. 4a shows a preferred variant of a cutting profile, seen in cross-sectional view, as formed in a recess of a cleaning swab as shown in FIGS. 1-3, whilst FIG. 4b shows the manner in which a strip of starting material is rolled up into a handle of the cleaning swab of FIGS. 1-3; and

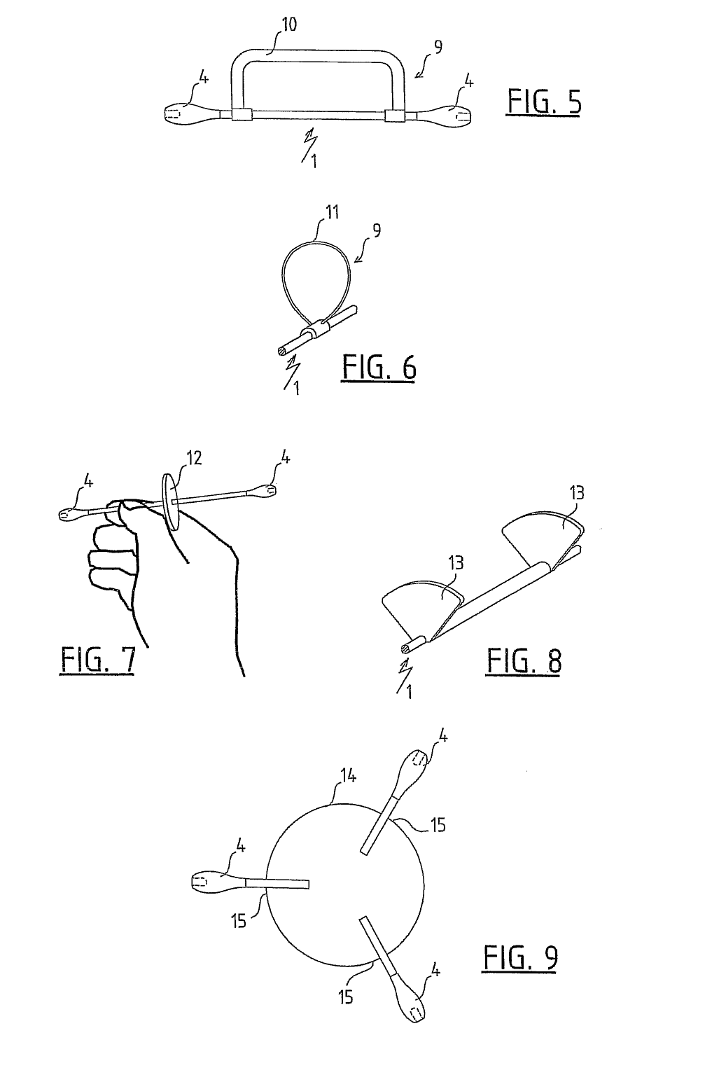

FIGS. 5-9 are schematic views of a cleaning swab with various types of stop surfaces according to the invention.

FIG. 1 shows a cleaning swab 1 comprising a handle made up of two handle segments in the form of tubular segments 2,3, which are connected at their facing ends by means of a suitable glue. The handle segment 3 is made of a softer material than the handle segment 2, as the handle segment 3 is inserted deeper into the ear in use, and injury to the ear must be prevented. For additional safety, the front edges of the handle segment 3 are provided with a rounded portion R. An egg-shaped thickened portion 4 of an absorbent cotton is provided round the illustrated end of the cleaning swab 1, for example by being glued thereto by means of a suitable glue. As FIG. 1 shows, the absorption material covers the front edge 5 of a recess 6 formed by an internal channel 7 in the handle segments 2,3 and extends into said recess 6.

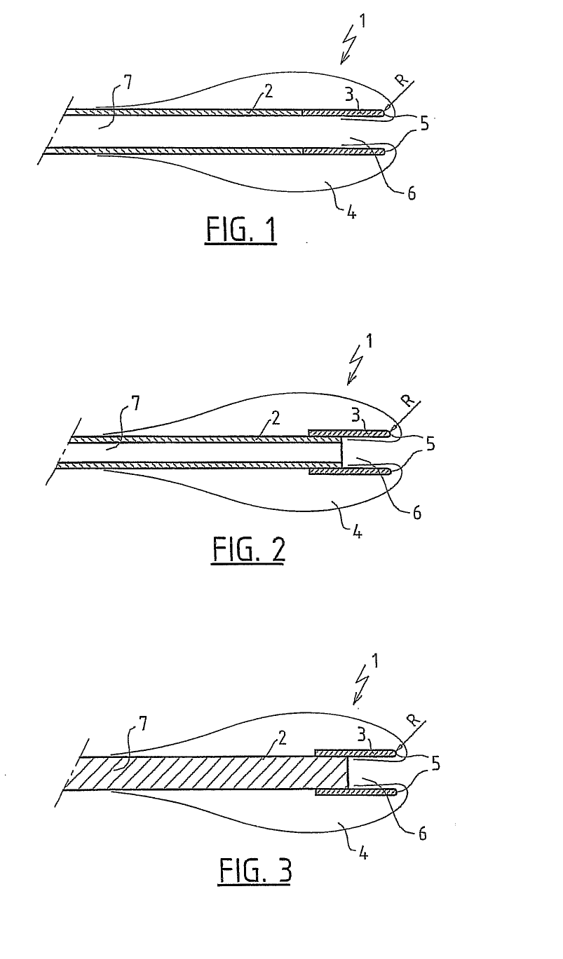

FIG. 2 corresponds to FIG. 1, and parts corresponding to parts shown in FIG. 1 are indicated by the same numerals. FIG. 2 shows the handle segment 3 partially moved over the handle segment 2, preferably being connected thereto by means of a suitable glue.

FIG. 3 corresponds to FIG. 2, and parts corresponding to parts shown in FIG. 2 are indicated by the same numerals. In this embodiment the handle segment 2 is a solid segment, so that this embodiment does not have an internal channel 7. By alternately pushing the cleaning swab of FIG. 1, 2 or 3 into the ear canal and rotating it, ear wax can be transferred to the recess 6 in an efficient manner, whilst damage to the ear canal is prevented in that the front edge 5 of the recess 6 is covered with absorption material.

FIG. 4a shows a cutting profile 8, seen in cross-sectional view, which can be formed in a recess 6 of the ear cleaning swab of FIG. 1, 2 or 3. The cutting profile 6 is a thread profile, for example, in the form of a cross as shown in FIG. 4a, which is connected to the walls of the recess 6 in said recess, for example by the application of a suitable glue or by means of locally provided holes or cuts. As a result, earwax is transferred to the recess 6 by carrying out a certain cutting or scraping movement and thus removed from the ear canal.

The handle of the ear cleaning swab 1 shown in FIG. 4b is made of a strip of a starting material, which strip is provided with weakened portions, in this case for example perforations, near the longitudinal edge thereof. The handle consisting of the two handle segments 2,3 is formed by subsequently rolling up said strip. The handle segment 3 will deform more easily than the handle segment 2 because of the presence of the perforations.

FIGS. 5-9 show various views of an ear cleaning swab 1 with various types of stop surfaces 9, whereas the cleaning swab 1 itself is not shown in detail lest so as not to make said figures unnecessarily complicated. The stop surfaces 9 function to prevent the ear cleaning swab 1 from being inserted (too) deep into the ear. In FIGS. 5, 6 and 7 the stop surface 9 is shown in schematic, perspective view as a bow 10, a flexible loop 11 and a disc 12, respectively, preferably being detachable in all three cases, in particular being snap-connected to the handle of the ear cleaning swab 1. FIG. 8 relates to a two-tip ear cleaning swab 1, that is, an ear cleaning swab 1 provided with a recess 6 and a thickened portion 4 of cotton at both ends, with the cotton covering the front edges 5 of the recesses 6. In this figure the stops 9 are shown in schematic, perspective view as detachable wings 13, which are in particular snap-connected to the handle of the ear cleaning swab 1 in this embodiment as well. FIG. 9 shows a schematic front view of a circular holder 14 with three ear cleaning swabs 1 attached thereto, with parts 15 of the circular circumference functioning as stops 9.

It is noted that the invention is not limited to the embodiments as shown herein, but that it also extends to other preferred variants that fall within the scope of the appended claims.

The orientation of the direction in which the snap connection is made is not limited to the orientation that is shown in the figures. Furthermore, the stop surfaces 9 may include any desired angle with the horizontal, i.e. an angle between 0 and 360 degrees.

1. A cleaning swab comprising a handle which is provided at at least one end thereof with a thickened portion consisting of absorption material, wherein the handle includes a recess near the end provided with the thickened portion, with the absorption material covering the front edge of the recess, leaving the entrance to the recess open, wherein the handle comprises at least two handle segments, and wherein the handle segment that faces towards the thickened portion deforms more easily than the handle segment that faces away from the thickened portion.

2. A cleaning swab according to claim 1, wherein the absorption material extends over the front edge and into the recess.

3. A cleaning swab according to claim 1, wherein the absorption material comprises at least one material selected from the group consisting of cotton, wool, paper and plastic.

4. A cleaning swab according to claim 1, wherein the handle is provided with an internal channel that forms the recess at the end of the handle provided with the thickened portion.

5. A cleaning swab according to claim 1, wherein the handle segments are made of different materials, and wherein the material of the handle segment that faces towards the thickened portion deforms more easily than the material of the handle segment that faces away from the thickened portion.

6. A cleaning swab according to claim 5, wherein the handle segments are glued together.

7. A cleaning swab according to claim 1, wherein the handle segment that faces towards the thickened portion is provided with weakened portions.

8. A cleaning swab according to claim 7, wherein said weakened portions are formed by perforations or cuts.

9. A cleaning swab according to claim 7, wherein the handle is made of a strip of a starting material, and wherein said strip can be converted from a flat starting position into a non-flat position of use.

10. A cleaning swab according to claim 1, wherein said recess is provided with a cutting profile.

11. A cleaning swab according to claim 1, wherein the handle comprises a stop surface that functions to prevent the handle from being inserted to deep into a body cavity.

12. A cleaning swab according to claim 11, wherein said stop surface is detachably connected to the handle.

13. A cleaning swab according to claim 1, wherein the handle is provided with a recess and with a thickened portion of absorption material near both ends, with the absorption material covering the front edges of the recesses, leaving the entrance to the recesses open.

14. A cleaning swab according to claim 13, wherein the absorption material extends over the front edge and into the recess.
